(19) United States
(12) Patent Application Publication
(10) Pub. No.: US 2009/0131641 A1
(43) Pub. Date: May 21, 2009

(54) Method for assembling subunits into capsoids

(75) Inventor: Michael Thies, Wuerzburg (DE)

Correspondence Address: KANESAKA BERNER AND PARTNERS LLP, 1700 DIAGONAL RD, SUITE 310, ALEXANDRIA, VA, 22314-2848, US

(73) Assignee: RESPONSIF GMBH

(21) Appl. No.: 12/289,931

(22) Filed: Nov. 7, 2008

Related U.S. Application Data
(63) Continuation of application No. 10/514,736, filed on Jan. 4, 2005, now abandoned.

(30) Foreign Application Priority Data
May 29, 2002 (DE) 102 24 111.2

(51) Int. Cl. C07K 14/00

(52) U.S. Cl. 530/402

(57) ABSTRACT

The invention relates to a method for assembling subunits forming capsoides in a solution containing a reduction agent in order to obtain capsoides. Initially, the reduction agent is inactivated or removed from the solution, subsequently, the ionic strength in the solution is increased to such an extent by adding at least one salt to the solution, that the subunits are assembled into capsoides.

The invention relates to a method for assembling subunits in a solution containing a reducing agent to give capsoids. Capsoids may be used as vehicles for transporting active compounds, in particular nucleic acids, into the interior of cells.

Capsoid-forming subunits, also called capsomers, are generally known to be able to form aggregates when a particular ionic strength or calcium ion concentration of the solution surrounding them is exceeded. The subunits are further known to oxidize with the formation of disulfide bridges between said subunits and to be able to form aggregates in the process. However, the subunits aggregate to capsoids only under certain conditions.

Chen, X. S., et al., Molecular Cell 5 (2000), pages 557 to 567, disclose that reducing the pH causes protein L1 of the human papilloma virus 16, expressed in E. coli, to assemble. Protein L1 forms pentamers as capsomers, 72 of which combine in each case to form a capsoid at pH 5.2. The disadvantage here is that the capsoids formed are irregular and that the combining does not proceed quantitatively, i.e. there remain free pentamers.

Braun, H., et al., Biotechnol. Appl. Biochem. 29 (1999), pages 31 to 43, disclose the assembly of capsoids from subunits recombinantly produced in E. coli. The subunits here consist of polyoma virus protein VP1. After expression, the VP1 proteins are isolated as pentamers from E. coli in a solution which has a low salt concentration and contains a reducing agent, and are stored. The reducing agent here prevents the formation of irregular aggregates, caused by the formation of disulfide bridges between the pentamers, during isolation and storage. Since the pentamers assemble in solutions of high ionic strength likewise into irregularly formed aggregates, the low salt concentration prevents the formation of such aggregates. In order to achieve assembly into regular capsoids, i.e. capsoids having a regular structure composed of 72 pentamers, the reducing agent is slowly removed, while the ionic strength is slowly increased. Both processes are carried out simultaneously by means of a dialysis over a period of from 5 to 7 days. Disadvantageously, this method which is referred to as dialysis method hereinbelow is very time-consuming.

DE 199 30 676 A1 discloses suppressing a structure-changing oxidation of proteins by using thiol reagents such as, for example, 2-mercaptoethanol or cysteine. An enzyme activity which has been reduced thereby can be virtually completely restored by removing 2-mercaptoethanol by means of dialysis or gel filtration.

It is the object of the invention to provide a method for assembling regular capsoids and a kit for carrying out said method, which method and kit do not have the disadvantages of the prior art. In particular, the method is intended to be carried out more rapidly than the dialysis method.

This object is achieved by the features of claims 1 and 17. Expedient embodiments result from the features of claims 2 to 16, 18 and 19.

The invention provides for a method for assembling capsoid-forming subunits in a solution containing a reducing agent to give capsoids, in which method the reducing agent is initially inactivated or removed from the solution and the ionic strength in said solution is then increased by adding at least one salt to said solution. In the process, the ionic strength is increased to at least such an extent that said subunits assemble to give said capsoids. The level to which the ionic strength has to be increased depends on the type of the subunits. The ionic strength at which capsoids form is known for the known capsoid-forming subunits. However, since the formation of the capsoids can also be readily monitored by an increase in light scattering, the increase in ionic strength required for inducing capsoid assemblage can also be easily determined.

For the purpose of the invention solution also means a colloidal solution or a suspension. The reducing agent may be dithiothreitol (DTT), β-mercaptoethanol, glutathione, dithioerythritol, cysteine, an SH group or a 2-mercaptoethane sulfonate sodium salt. In order to obtain stable capsoids, the reducing agent is inactivated or removed from the solution. This may cause the formation of intracapsomeric, in particular intrapentameric, disulfide bridges which, due to subsequent conformational changes, have a stabilizing effect on the intercapsomeric, in particular interpentameric, interactions. Inactivating the reducing agent means that it is treated in such a way that it can no longer prevent oxidation of SH groups present in the subunits by an oxidizing agent present in the solution, such as, for example, atmospheric oxygen dissolved therein, to give disulfide bridges. This may occur very rapidly, for example by specific degradation or oxidation of the reducing agent. Removal of the reducing agent may likewise occur very rapidly, for example by a chromatographic method, in particular by means of a commercially available desalting column. The reducing agent need only be removed to such an extent that formation of disulfide bridges is enabled by oxidation of SH groups present in the subunits by an oxidizing agent present in the solution, such as, for example, dissolved oxygen. Quantitative removal of the reducing agent is not required for this purpose. The ionic strength in the solution containing the subunits needs to be so low that merely inactivating or removing the reducing agent does not yet produce any capsoids. This means that the ionic strength in the solution containing the subunits may also be higher initially, if the ions responsible therefore are removed together with the reducing agent.

The salt to be added to the solution in order to increase the ionic strength may be added in solid form or as a solution. After adding the salt, the solution in which the capsoids can form should be incubated. The incubation is preferably carried out at room temperature for about 30 min.

The entire process may be finished after 1 to 2 hours. Surprisingly, it was found that the capsoids formed in the process, despite their rapid formation by merely increasing the ionic strength, have the same regular structure as the capsoids obtained by the very time-consuming dialysis method. The method has the advantage that it is possible to more accurately predetermine and control the conditions under which assembly takes place, such as, for example, ionic strength, pH, temperature or protein concentration, than in the dialysis method. This is crucially important with regard to applying the method of the invention to packaging an active compound into capsoids. In fact, the capsoids form in the above-described dialysis method as soon as a particular salt concentration is reached and the concentration of the reducing agent has fallen below a particular level. However, it is not possible to form the capsoids with particular predetermined conditions, for example conditions favorable to the active compound to be packaged therein. The method of the invention, in contrast, enables conditions favorable to the active compound to be set, as long as the subunits still assemble into the capsoids.

Another advantage of the method of the invention over the dialysis method is the possibility of also packaging an active compound, which is sensitive to the reducing agent, into the capsoids by adding the active compound only after removing or inactivating the reducing agent.

The method furthermore has the advantage that it is possible to avoid contacting the solution and the active compound which may or may not be present therein with a dialysis membrane. Otherwise, unspecific bindings may arise at the dialysis membrane. This would result in a loss of active compound and subunits. The method furthermore makes it possible to package small active compound molecules into capsoids, which would pass through the dialysis membrane in the dialysis method. In order to prevent such active compound molecules from being removed together with the reducing agent, they are preferably added to the solution only after removing said reducing agent.

Another advantage of the method of the invention is the fact that it is easier to maintain sterile conditions than in the dialysis method, due to the smaller amount of liquids to be handled alone.

In a preferred embodiment of the method, the reducing agent is removed by means of size exclusion chromatography or dialysis or inactivated by means of an oxidizing agent which oxidizes essentially only the reducing agent. More specifically, the oxidizing agent is chosen such that its redox potential is not sufficient for oxidizing the SH groups in the subunits. The following combinations of oxidizing and reducing agents have proved particularly advantageous here: oxidized glutathione-reduced glutathione, cystine-cysteine, cystamine-cysteamine, di(2-hydroxyethyl) disulfide-β-mercaptoethanol.

Size exclusion chromatography has the advantage of being able to be carried out very rapidly. Another advantage is the fact that the method can be transferred very efficiently from a laboratory process on a small scale to a process for producing relatively large amounts of capsoids. Since the method of the invention requires only the generally low molecular weight reducing agents to be removed, this process can also be carried out rapidly by dialysis, however.

The ionic strength may be increased step by step. This may further increase the homogeneity of the capsoids. It has proved advantageous to increase the ionic strength in 5 equally large steps. The time interval between the steps is preferably about 10 minutes. After the last step, an incubation at room temperature for approx. 10 minutes is advantageous.

The ionic strength I may be increased to a value of no more than 1.5 mol/l, in particular no more than 1 mol/l, preferably no more than 0.75 mol/l. Here, I=0.5*Σcᵢ*zᵢ², where cᵢ is the concentration and zᵢ the charge of the ions i in the solution.

Preferably, the total protein concentration in the solution, due to the capsoids and the subunits, does not fall below 15 μg/ml, in particular 150 μg/ml, preferably 225 μg/ml. This enables a higher yield and regularity of the capsoids to be achieved during assembly. The higher the concentration of the subunits in the solution, the more rapidly assembly takes place.

In an advantageous embodiment of the method, the subunits consist of recombinantly produced proteins or peptides. This is particularly advantageous if large amounts of the capsoids are to be prepared. There is no need to use any native, usually in the form of capsids, and possibly infectious starting material which would first have to be disassembled prior to assembly. The subunits preferably comprise the viral protein “VP1” of a polyoma virus, the viral protein “L1” of a papilloma virus, the “core protein”, together with the “membrane protein” and the “envelope protein”, of the flavi virus, the “core protein” of the hepatitis B virus or of the hepatitis C virus, the viral protein “VP1” of the SV40 virus, the viral protein “gag” of the HI virus, the viral protein “VP5” of the herpes simplex virus, the viral protein “lambda1”, “lambda2” or “lambda3” of the reo virus or the “capsid protein” of the Norwalk virus. These viral proteins are particularly suitable for preparing capsoids for packaging active compounds.

In an advantageous embodiment of the method, SH groups present in the subunits are oxidized after the assembly to give the capsoids. This causes the formation of intracapsomeric, in particular intrapentameric, disulfide bridges and, due to the conformational change induced thereby, stabilization of the capsoids. The conditions here are advantageously chosen so as to prevent the formation of disulfide bridges between the capsomers, in particular pentamers. Particularly advantageously, the SH groups are oxidized by adding an oxidizing agent, in particular cystine, cystamine, di(2-hydroxyethyl) disulfide or oxidized glutathione (GSSG). Incubation with about 7 mmol/l oxidized glutathione at room temperature for about 30 minutes has proven advantageous. The oxidizing agent may subsequently be removed, for example by dialysis or a chromatographic method, or inactivated by adding a reducing agent.

The solution may contain an active compound when the ionic strength is increased. In the process, capsoids containing said active compound may form, which may serve as vehicles in order to introduce said active compound into cells. It is also possible to add the active compound to the solution only after the reducing agent has been removed or inactivated. This is particularly advantageous if the active compound is sensitive to the reducing agent. The active compound may be a substance acting inside cells, in particular a nucleic acid, a protein, an antibody, a peptide, an enzyme, a transcription factor, a phosphorothioate-derivatized oligonucleotide, PNA, a chimera of PNA and DNA, a DNA-peptide complex or a low molecular weight active compound. The active compound may be coupled to or associated with at least one of the subunits. This enables the active compound to be transported specifically to a predefined region within an eukaryotic cell. Coupling or associating methods are disclosed in WO 00/00224. Preferably, the active compound is coupled to or associated with the subunit in such a way that it is located on the inside of the capsoids after assembly. The inclusion of the active compound in the capsoids causes improved absorption of the active compound into the cell.

In one embodiment of the method, the assembled capsoids are lyophilized. This is particularly advantageous if the capsoids contain an active compound which, in its dissolved form, may be degraded with time or may otherwise lose its efficacy. Since packaging into the capsoids may also be carried out much more rapidly than in the dialysis method, the lyophilization of capsoids produced according to the invention may also prevent degradation or loss of efficacy of such an active compound earlier and thus better than in the case of capsoids which have been produced using the conventional method.

The invention further relates to a kit for carrying out the method of the invention, comprising
capsoid-forming subunits in a solution containing a reducing agent and
an oxidizing agent suitable for inactivating the reducing agent, which oxidizes essentially only said reducing agent.

The kit may also comprise a salt for increasing the ionic strength. The oxidizing agent and the salt may be present in a predetermined amount and/or dissolved at a predetermined concentration.

Exemplary embodiments of the invention are illustrated in the drawing and are explained in more detail in the following description. The figures show:

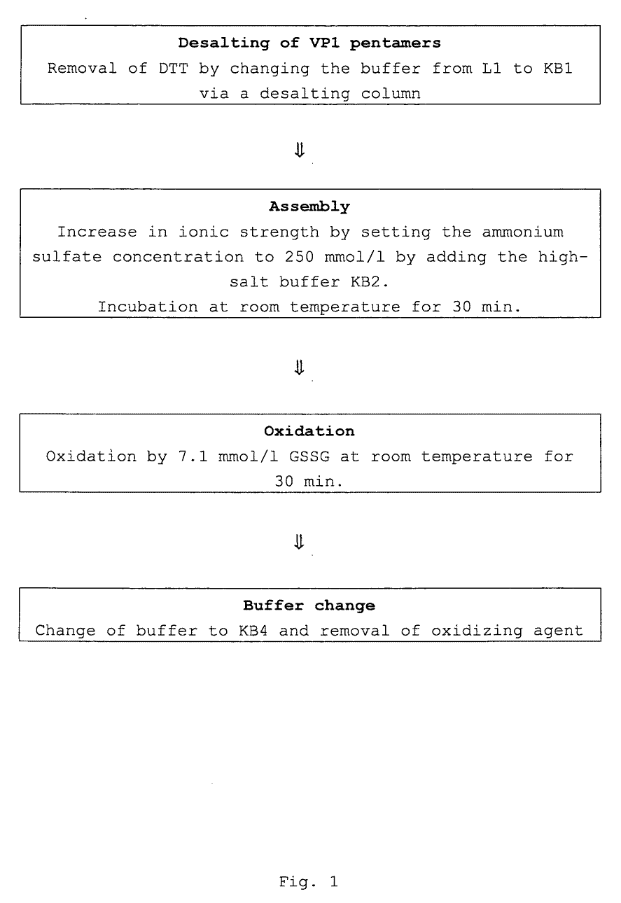

FIG. 1 is a diagrammatic representation of a method of the invention,

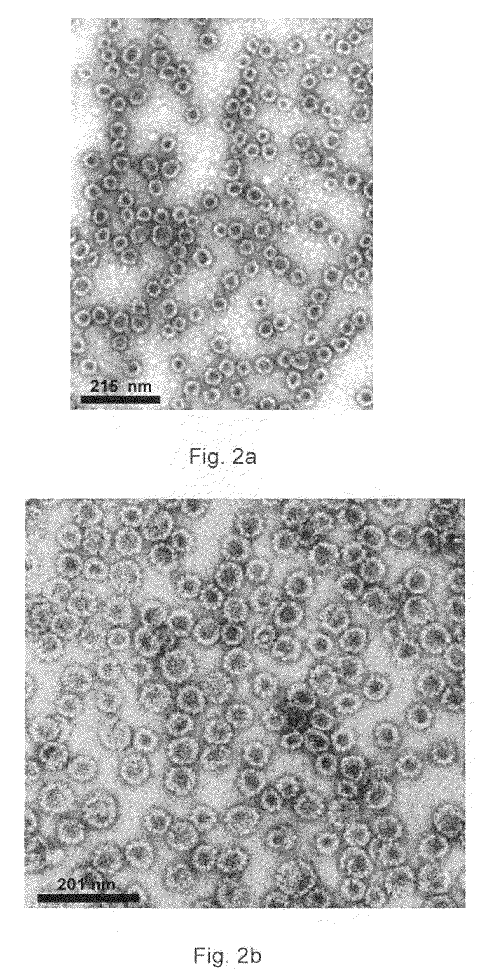

FIG. 2a, b each depict an electron microscopy image of capsoids produced from the viral protein VP1 according to the method of the invention and according to the conventional method,

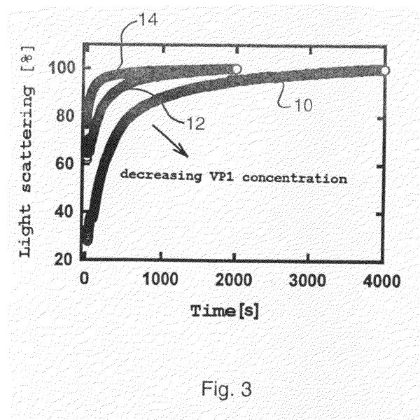

FIG. 3 is a graphic representation of light scattering caused by capsoids produced according to the invention as a function of time, after addition of the salt,

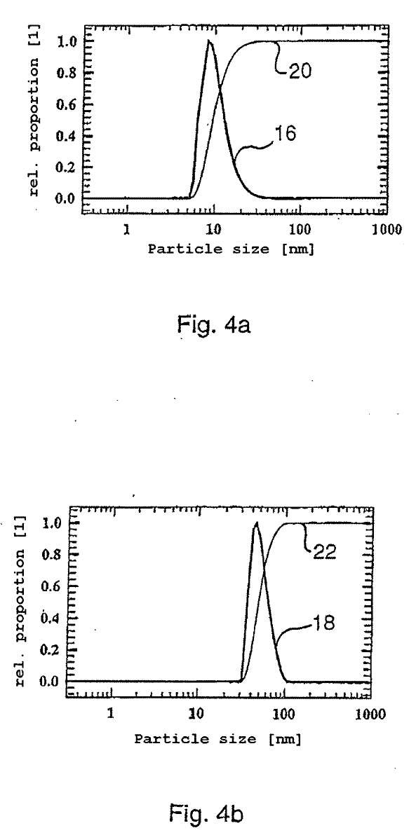

FIG. 4a, b is a graphic representation of in each case one analysis of the particles present in the solution before and after addition of the salt by means of photon correlation spectroscopy (PCS) and

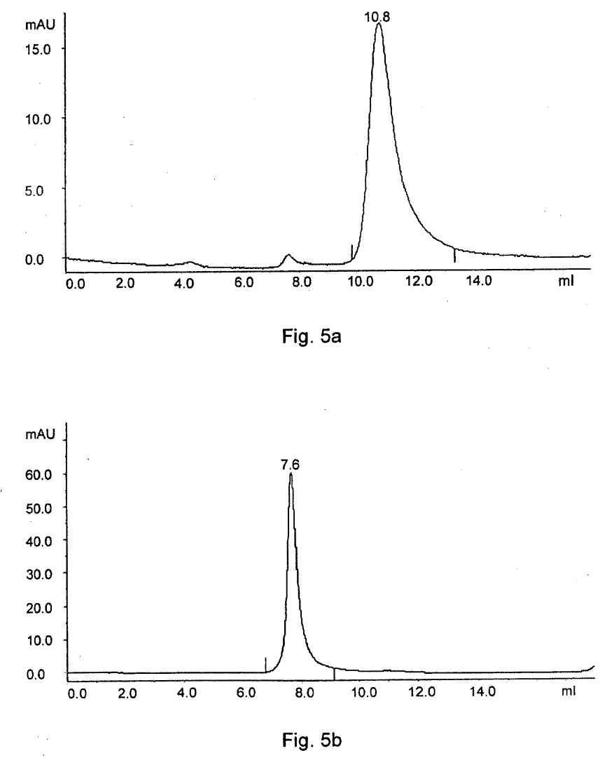

FIG. 5a, b is a graphic representation of in each case one analytical gel filtration of the particles present in the solution before and after addition of the salt.

The polyoma virus envelope protein VP1 is obtained recombinantly as a homopentamer from E. coli and purified chromatographically. After purification, it is present in the “L1 buffer” (50 mmol/l sodium phosphate, 150 mmol/l NaCl, 2 mmol/l EDTA, 5% glycerol, 6 mmol/l DTT, pH 7.0) under reducing conditions. FIG. 1 depicts diagrammatically the course of the method of the invention carried out therewith. In order to obtain stable VP1 capsoids by the method of the invention (intrapentameric disulfide bridges are formed which, due to subsequent conformational changes, have a stabilizing effect on the interpentameric interactions), it is necessary to remove beforehand the reducing agent DTT from the VP1-pentamer solution. This is carried out with the aid of a commercially available desalting column such as “PD10” for analytical or “HiPrep 26/10 Desalting” for preparative reaction mixtures, both of which are available from Amersham Biosciences. The mobile phase used is 50 mmol/l sodium phosphate, 150 mmol/l NaCl, 2 mmol/l EDTA, 5% glycerol, pH 6.8 (referred to as KB1 buffer hereinbelow). Due to the low pore size of the column material, the protein is in the flow-through, while the reducing agent, due to its smaller size, elutes from the column with a delay. The protein is readily diluted by this procedure. The VP1 concentration should not fall below 250 μg/ml. As an alternative to the method described, the reducing agent may also be removed by means of dialysis against the KB1 buffer.

The assembly of the VP1 pentamers is induced by adding a high-salt buffer. To this end, the volume of the VP1 solution used must be known in order to be able to set the exact ionic strength for assembly. The assembly buffer KB2 (10 mmol/l Tris/HCl, 150 mmol/l NaCl, 5% glycerol, 3 mol/l ammonium sulfate, pH 8.0) is then diluted by adding it to the protein solution in a 1:12 ratio (1 part of KB2, 11 parts of protein solution) and mixed. After this step, the final concentration of ammonium sulfate is 250 mmol/l. This corresponds to an ionic strength of 750 mmol/l. This is followed by an incubation phase of 30 minutes at room temperature, during which capsoid formation is completed. In order to further increase the homogeneity of the capsoids, the assembly step may be modified such that the high-salt buffer is added to the protein solution in several steps. Five equally large steps with time intervals of 10 minutes increase the ammonium sulfate concentration step-by-step to 250 mmol/l. This procedure is then completed by incubating at room temperature for 10 minutes.

The VP1 capsoids obtained are subjected to oxidative conditions for stabilization by means of oxidized glutathione. This results in the formation of intrapentameric disulfide bridges. For this purpose, the protein solution is admixed with the KB3 buffer (10 mmol/l Tris/HCl, 150 mmol/l NaCl, 5% glycerol, 3 mol/l ammonium sulfate, 400 mmol/l GSSG, pH 8.0) in a 1:56 ratio (1 part of KB3, 55 parts of protein solution; final GSSG concentration 7.1 mmol/l) and then incubated at room temperature for 30 minutes.

In the last step, capsoids formed are dialyzed against PBS buffer (PBS Dulbecco, Biochrom) containing 0.7 mmol/l CaCl₂ (KB4). This removes the oxidizing agent. The protein may be concentrated to the desired concentration via diafiltration or precipitation.

FIGS. 2a and 2b depict in each case an electron microscopy image of VP1 capsoids. The capsoids depicted in FIG. 2a have been produced by means of the method of the invention. The capsoids depicted in FIG. 2b have been formed by means of the conventional method in which the ionic strength is increased by dialysis with simultaneous removal of the reducing agent. FIGS. 2a and 2b demonstrate that the capsoids produced by the method of the invention have the same quality as the conventionally produced capsoids. They have a regular shape and a diameter of about 40 nm.

The capsoids, due to their larger diameter compared to the pentamers, scatter light irradiated into the solution more strongly than the pentamers. The extent of light scattering of the solution may therefore be used as a measure of the capsoid content of the solution, thus enabling the course of capsoid formation to be monitored therewith. FIG. 3 depicts the graphic representation of light scattering caused by capsoids produced according to the invention as a function of time, after addition of the salt. The curves 10, 12 and 14 correspond to the VP1 concentrations 194 μg/ml, 387 μg/ml and 775 μg/ml. This indicates that the capsoids form more rapidly with increasing VP1 concentration. Thus, said formation may be completed after a few minutes at a high VP1 concentration.

The size of the particles formed may also be determined by means of photon correlation spectroscopy (PCS). This makes use of the fact that particles move in a solution more slowly with increasing size. In FIGS. 4a and 4b, the curves 16 and 18 depict in each case the proportion of particles of a particular size relative to the total number of particles, said proportion being determined by means of PCS, with the units being chosen arbitrarily. The curves 20 and 22 are in each case integration curves of curves 16 and 18. FIG. 4a depicts the result of a PCS measurement carried out prior to assembly. The peak of curve 16 results from the pentamers having a diameter of about 10 nm. FIG. 4b depicts the result of a PCS measurement after assembly of the pentamers. The peak of curve 18 is caused by the capsoids formed which have a diameter of about 40 nm. It shows a distinct shift compared to the peak of curve 16 of FIG. 4a. The comparison of curves 16 and 18 indicates that the pentamers have been assembled completely into capsoids. The peak of curve 18, which is comparatively narrow for a measurement by means of PCS, moreover reveals that the capsoids have a uniform size.

Analytical gel filtration has also been carried out. The protein content of the eluate has been determined by measuring UV absorption at a wavelength of 280 nm. FIG. 5a depicts the result determined for VP1 pentamers and FIG. 5b depicts the result determined for the capsoids formed therefrom, with in each case arbitrary units (mAU) having been chosen for absorption. The VP1 pentamers generate a peak at a retention volume of 10.82 ml. This peak is no longer visible in FIG. 5b. This suggests that the VP1 pentamers have completely assembled into capsoids. The narrow peak caused by the capsoids formed, which appears at a retention volume of 7.66 ml, in FIG. 5b indicates that the capsoids have a uniform size.

1. A method for assembling capsoid-forming subunits in a solution containing a reducing agent to give capsoids, in which method the reducing agent is initially inactivated or removed from the solution and the ionic strength in said solution is then increased, by adding at least one salt to said solution, to at least such an extent that said subunits assemble to give said capsoids,
wherein the ionic strength of the solution is increased in steps.

2. The method as claimed in claim 1, in which the reducing agent is removed by means of size exclusion chromatography or dialysis or is inactivated by means of an oxidizing agent which oxidizes essentially only said reducing agent.

3. The method as claimed in claim 1, in which the ionic strength is increased in 5 steps that are equal in size.

4. The method as claimed in claim 1, in which there is a time interval of about 10 minutes between the steps.

5. The method as claimed in claim 1, in which the ionic strength I is increased to a value of no more than 1.5 mol/l.

6. The method as claimed in claim 1, during which the total protein concentration in the solution, due to the capsoids and the subunits, does not fall below 15 μg/ml.

7. The method as claimed in claim 1, in which the subunits consist of recombinantly produced proteins or peptides.

8. The method as claimed in claim 1, in which the subunits comprise the viral protein “VP1” of a polyoma virus, the viral protein “L1” of a papilloma virus, the “core protein”, together with the “membrane protein” and the “envelope protein”, of the flavi virus, the “core protein” of the hepatitis B virus or of the hepatitis C virus, the viral protein “VP1” of the SV40 virus, the viral protein “gag” of the HI virus, the viral protein “VP5” of the herpes simplex virus, the viral protein “lambda1”, “lambda2” or “lambda3” of the reo virus or the “capsid protein” of the Norwalk virus.

9. The method as claimed in claim 1, in which SH groups present in the subunits are oxidized after the assembly to give the capsoids.

10. The method as claimed in claim 9, in which the SH groups are oxidized by adding an oxidizing agent, in particular cystine, cystamine, di(2-hydroxyethyl) disulfide or oxidized glutathione.

11. The method as claimed in claim 1, in which the solution comprises an active compound when the ionic strength is increased.

12. The method as claimed in claim 11, in which the active compound is added to the solution only after the reducing agent has been removed or inactivated.

13. The method as claimed in claim 11, in which the active compound is a substance acting inside cells, in particular a nucleic acid, a protein, an antibody, a peptide, an enzyme, a transcription factor, a phosphorothioate-derivatized oligonucleotide, PNA, a chimera of PNA and DNA, a DNA-peptide complex or a low molecular weight active compound.

14. The method as claimed in claim 11, in which the active compound is coupled to or associated with at least one of the subunits.

15. The method as claimed in claim 14, in which the active compound is coupled to or associated with the subunit in such a way that, after the assembly, it is located on the inside of the capsoids.

16. The method as claimed in claim 1, in which the capsoids are lyophilized.

17. A kit for carrying out a method as claimed in claim 1, which kit comprises capsoid-forming subunits in a solution containing a reducing agent and an oxidizing agent suitable for inactivating the reducing agent, which oxidizes essentially only said reducing agent and a salt for increasing the ionic strength.

18. The kit as claimed in claim 17, in which the oxidizing agent is present in a predetermined amount and/or dissolved at a predetermined concentration.

19. The kit as claimed in claim 17, in which the salt is present in a predetermined amount and/or dissolved at a predetermined concentration.
